(19) United States
(12) Patent Application Publication
(10) Pub. No.: US 2009/0263317 A1
(43) Pub. Date: Oct. 22, 2009

(54) METHOD OF SCREENING THE ACTIVITY OF THE SMOOTHENED RECEPTOR TO IDENTIFY THERAPUTIC MODULATION AGENTS OR DIAGNOSE DISEASE

(76) Inventors: Wei Chen, Chapel Hill, NC (US); Lawrence S. Barak, Durham, NC (US); Marc G. Caron, Hillsborough, NC (US); Robert Lefkowitz, Durham, NC (US)

Correspondence Address: PASSE' INTELLECTUAL PROPERTY, LLC, 1717 BRASSFIELD RD., RALEIGH, NC, 27614, US

(21) Appl. No.: 12/097,697

(22) PCT Filed: Dec. 14, 2006
(86) PCT No.: PCT/US06/62102

§ 371 Date: Jan. 26, 2009

Related U.S. Application Data
(60) Provisional application No. 60/750,482, filed on Dec. 15, 2005.

(51) Int. Cl. A61K 51/00; C12Q 1/48; A61K 49/00

(52) U.S. Cl. 424/1.11; 435/15; 424/9.1

(57) ABSTRACT

The invention relates to a screening method for the Smoothened receptor for testing compositions as potential Smoothened receptor ligands, either agonist or antagonist activity, by use of an arrestin-reporter molecule conjugate. It also relates to testing cells and individuals by administering a smoothened receptor ligand-reporter molecule conjugate and observing locations where the ligand binds and then using an increased number of surface Smoothened receptors compared to a pre-established criteria as an indication of a cancerous growth or tumor.

### FEDERALLY SPONSORED RESEARCH

This invention was made with Government support under: supported in part by NIH grant HL 16037 (R.J.L.), and HL 61365 (L.S.B.). The Government has certain rights to this invention.

This application is a non-provisional application of provisional application 60/750,482 filed Dec. 15, 2005.

### BACKGROUND OF THE INVENTION

1. Field of the Invention

The present invention relates to a method of screening receptor activity with test ligand samples for therapeutic activity and to screen individuals and cells for diagnostic purposes. In particular, the invention relates to screening the Smoothened receptor for the effects of test ligand compositions on the activity of the receptor. It also relates to testing cells and individuals and the receptor as an indication of a disease state, especially for cancer.

2. Description of the Related Art

The Hedgehog (Hh) proteins are known as a family of signal molecules that can act as mediators in the developmental processes such as growth and patterning, for both invertebrates and vertebrates. It is known that changes in the Hh pathway can lead to birth defects and in adult cells can lead to cancer. While the extent to which Hh participates and controls the growth of cancer cells is not completely known, it is already known that cancer related to brain, skin, muscle stomach, pancreas, lung, prostate and bladder all involve the Hh pathway. Nature Vol 432, Nov. 18, 2004 pgs 324-331. Two transmembrane protein receptors, Patched (Ptc) and Smoothened (Smo) mediate the responses to the Hh proteins. Ptc, a 12 transmembrane protein regulates (inhibits) the activity of the Smo protein, a 7 transmembrane protein similar in structure to a Frizzled protein of the Wnt family rather than the well known 7 transmembrane G Protein Coupled Receptors (GPCRs). The Smo proteins appear to be involved in embryonic pattern formation and in stem cell renewal, tissue repair and regeneration unlike the GPCR family of proteins. When not inhibited by Ptc, Smo signals are transduced to Gli. When Hh binds to Ptc, it relieves the inhibitory control of Smo. For example, excess signaling of Smo when Ptc is blocked from inhibiting Smo activity is known to contribute to medulloblastoma while inhibition of Smo leads to an elimination of tumors. Cancer Res 2005; 65; (12) Jun. 15, 2005 pgs 4975-4978.

The Hh pathway in vertebrates is considerably more complex than in the well studied D. melanogaster. There are three Hh genes in mammals, sonic, Indian and desert hedgehog (Shh, Ihh and Dhh), two Ptc genes (Ptc1 and Ptc2) and three Gli homologues (Gli1, Gli2 and Gli3). Nature Reviews, volume 6, April 2005 pages 307-317.

In the situation where there is excess Hh (for example, excess Shh), the Ptc carries inhibiting mutations or the Smo inappropriately signals, there appears to be conditions that lead to several forms of cancer, especially in vertebrates. It appears that Ptc normally acts to keep cell proliferation in check by keeping Smo in check by modulating its signaling, specifically though Smo inhibition. It also appears that loss of Hh signaling, which could result in essentially permanent inhibition of Smo, also creates metabolic problems and appears to result in cyclopia and other developmental defects of the face, forebrain and other organs and structures.

Compositions and assays are known which act as agonists or antagonists in the Hh pathway, including with the Smo receptor. In U.S. Pat. No. 6,492,139 to de Sauvage, there is disclosed novel homologues of Smo as well as the sequence of both human and rat Smo. There are also described several antibodies to vertebrate Smo. In U.S. Pat. No. 7,115,653 to Baxter there is disclosed compounds which correct or inhibit an aberrant or unwanted growth state by antagonizing a normal Ptc pathway or agonizing a Smo or Hh activity.

A large number of regulators of the Hh pathway including the Smo function are disclosed in U.S. Pat. No. 7,098,196 and US Patent Application No US 2006/0128639 both to Beachy. These compounds are shown to modulate the Hh pathway and several utilities of such compounds are described in detail. The Beachy references are hereby incorporated by reference including the disclosure of the utility of compositions which involve control of Smo and the entire Hh pathway activity. The assays described in these disclosures for discovery of modulators involve reporter gene based assays which measure the end stage of the cascade of events, i.e. transcriptional modulation. A reporter gene construct is inserted into a reagent cell in order to generate a detection signal dependent on Ptc loss of function, Hh gain of function, Smo gain of function or stimulation by Hh itself. These signaling events though are difficult to follow in a timely manner using a reporter assay which may take many up to 2 days or more after the events have occurred to provide an appropriate readout. Additionally, because the readout is many biochemical steps past the point where a change occurs in Smo activity, reporter assays may falsely report changes in Smo activities that are upstream of the reporter assay but due to Smo downstream biochemical and biological events rather than Smo activities or Smo upstream activities. The related art of the Smo receptor discloses no other useful cell based assays for following the Hh pathway.

Methods for screening GPCRs in a direct manner, in a cell based assay, have been known for almost 10 years. In U.S. Pat. No. 5,891,646 to Barak there is disclosed a method for screening GPCRs using a conjugate of a beta-arrestin and a detectable (reporter) molecule. The receptor is shown to translocate between the cytosol and the cell membrane upon activation of the receptor by an agonist. It can also translocate between the cell membrane and the membrane of structures within the cytosol. There is no prior evidence that this assay has utility for any other receptor group as evidenced by its long standing acceptance and lack of reported utility for any other receptor group.

Activity-dependant internalization of Smoothened was disclosed as mediated by β-arrestin 2 and GRK2 in Science Vol 306 Dec. 24, 2004 pages 2257-2260 by inventors of the present technology. The authors of that paper postulated that this knowledge “may provide a platform” for a discovery assay however, until the present invention, the ability to successfully assay ligands with this information was unknown and speculative. There has been no disclosure of a diagnostic assay.

Accordingly, screening methods developed around reporting assays are cumbersome, at best, to employ and not ideal for either diagnostic applications or automated drug discovery where a lack of fidelity can add considerably to the expense and difficulty of screening the Hh pathway. Therefore, there is a need for a method to directly measure the Hh path that is both efficient and time effective and can be used to diagnose medical conditions.

### SUMMARY OF THE INVENTION

It has been discovered that compounds which modulate the Hh pathway can be determined by observing the response of the Smoothened receptor. In addition, the assay is quick accurate and real time in discovering that the Smoothened receptor is inhibited in a normal manner or is expressing in a manner likely to cause over proliferation or cancer or a condition involving under expression and lack of proliferation of cells.

In one embodiment of the invention, there is method for screening a test compound for Smoothened receptor activity comprising:
a. providing one or more cells that expresses a Smoothened receptor and contains a conjugate of arrestin and a reporter molecule, and also contains a kinase capable of phosphorylating the Smoothened receptor;
b. exposing the one or more cells to a test compound; and
c. determining if the test compound is active on the Smoothened receptor activity.

In another embodiment, the invention relates to the diagnosis of disease in the Hh pathway, specifically a method for diagnosing the cause of a medical condition in a test subject mediated by the Smoothened receptor wherein the medical condition is mediated by a deviation in the Smoothened receptor activity from a pre-established activity criteria comprising:
a. providing a cell from a test subject which has been transformed to contain a conjugate of arrestin and a reporter molecule and which cell contains a kinase capable of phosphorylating the Smoothened receptor for example GRK2;
b. exposing the cell to a known ligand of the Smoothened receptor;
c. determining the activity of the Smoothened receptor in the cell; and
d. comparing the activity in the cell to the pre-established activity criteria.

In yet another embodiment of the invention there is disclosed a diagnostic assay for determining if a cell is cancerous. Specifically, the method is a method for the diagnosis of cancer in a subject comprising:
a. administering to a subject or one or more cells from a subject, a conjugate of a ligand of the Smoothened receptor and a reporter molecule;
b. quantitatively determining the location or intensity of the reporter molecule in the subject or cell;
c. determining the presence of cancer where ever there is an accumulation of reporter molecule that is above a pre-established criteria.

These and other objects of the present invention will be clear when taken in view of the detailed specification and disclosure in conjunction with the appended figures.

### BRIEF DESCRIPTION OF THE DRAWINGS

FIG. 1—Localization of beta-arrestin2 green fluorescent protein (βarr2-GFP) to the plasma membrane in cells overexpressing Smo.

FIG. 2—Ptc and cyclopamine inhibit membrane recruitment of βarr2-GFP, and SAG relieves Ptc inhibition of recruitment of βarr2-GFP to the plasma membrane.

FIG. 3—Phosphorylation of Smo mediated by GRK2.

FIG. 4—Effects of GRK2, Ptc, SAG, and cyclopamine on Smo phosphorylation.

FIG. 5—Output State of the Smoothened Receptor and Arrestin Reporter with Smoothened in the inactivate state.

FIG. 6—Output State of the Smoothened Receptor and Arrestin Reporter with Smoothened in the activate state.

FIG. 7—Translocation of the Smoothened Receptor to a vesicle inside the cell.

### DETAILED DESCRIPTION OF THE INVENTION

The general description of the invention and how to use the assays are stated in the Brief Summary above. This detailed description defines the meaning of the terms used herein and specifically describes embodiments in order for those skilled in the art to practice the invention. The above interests in successfully assaying compositions for modulating the Hh pathway and in detecting a disease condition including cancer can readily be seen from the disclosure which follows and are met by the present invention. This detailed description defines the meaning of the terms used herein and specifically describes embodiments in order for those skilled in the art to practice the invention.

The term “arrestin” as used herein has its ordinary meaning in the art and is intended to encompass all types of arrestin, including but not limited to visual arrestin (sometimes referred to as Arrestin 1), βarrestin 1 (sometimes referred to as Arrestin 2), and βarrestin 2 (sometimes referred to as Arrestin 3).

The term “βarrestin” as used herein is intended to encompass all types of βarrestin, including but not limited to βarrestin 1 and βarrestin 2.

The term “reporter molecule” as used herein refers to molecules useful for detecting the location, intensity or quantity of other molecules that are attached to it, e.g. as a conjugate. These molecules are often called detectable molecules and such phrases are used interchangeably herein. Molecules detectable by spectroscopic, photochemical, biochemical, immunochemical, electrical, radiographic and optical means are known. Optically detectable molecules include fluorescent labels, such as commercially available fluorescein and Texas Red. Detectable molecules useful in the present invention include any biologically compatible molecule which may be conjugated to a βarrestin protein or to a Smoothened ligand without compromising the ability of βarrestin or the ligand to interact with the Smoothened system, and without compromising the ability of the reporter molecule to be detected. These include molecules which interact with other molecules as a means of creating a reportable event for example as some reporter molecules used in the known BRET and FRET assays which include fragmented molecular systems. Conjugated molecules (or conjugates) of βarrestin and detectable molecules as well as conjugates of a Smo ligand and a reporter molecule are thus useful in the present invention. Preferred for attachment to arrestin are detectable molecules capable of being synthesized in the cell to be studied (e.g., where the cell can be transformed with heterologous DNA so that the βarrestin-reporter molecule chimera is produced within the cell). Particularly preferred are those detectable molecules which are inherently fluorescent in vivo. Suitable detectable molecules must be able to be detected with sufficient resolution within a cell that translocation of βarrestin from the cytosol to the cell membrane in response to agonist or antagonist binding to Smoothened receptors can be qualitatively or quantitatively assessed. They also can be sufficient for detection of Smo from the cell membrane to an internal location or internal membrane. Molecules detectable by optical means are presently one important embodiment. Where accumulation of a Smo ligand and a reporter molecule are used to diagnose the location or size of a tumor, it is important to be able to detect the reporter molecule in the whole animal. Accordingly, embodiments of the reporter molecule for this use include radioactive isotopes or the like for indication location and intensity or quantity in the subject being tested.

As used herein, “Green Fluorescent Protein” refers to the various naturally occurring forms of GFP which can be isolated from natural sources, as well as artificially modified GFPs which retain the fluorescent abilities of native GFP. Various mutants of GFP have been created with altered excitation and emission maxima. Two characteristics of wild-type GFP which affect its usefulness in mammalian cell lines are the need to excite it at UV wavelengths to obtain a maximal fluorescent signal, and decreased fluorescence at temperatures over 23.degree. C. However, the S65T/GFP mutant overcomes these limitations. Additional alterations in the GFP protein sequence which provide inherently fluorescent, biologically compatible molecules will be apparent to those in the art; sequence alterations may be made to alter the solubility characteristics of the protein, its excitation wavelength, or other characteristics, while retaining useful fluorescent properties. See U.S. Pat. Nos. 7,138,240; 7,770,449; 6,110,693; and 5,891,646 and US Patent application Nos. 2004/0209308 and 2004/0101887 herein after referred to as the “Duke patents” and incorporated herein by reference.

The term “conjugate” as used herein refers to 2 or more proteins joined together in a fashion that they both retain their activity. For example, a conjugate of an arrestin and a reporter molecule allows the arrestin molecule to continue to be involved in the translocation pathways while the reporter molecule indicates its location or intensity. It also could for example be where a Smo ligand attached to a reporter molecule such that the ligand remains active and the reporter molecule remains detectable. See for example the “Duke patents” for a detailed description and examples of arrestin-reporter molecule conjugates.

The term “cell” as used herein refers to cells useful in the methods of the present invention including eukaryotic and prokaryotic cells, including but not limited to bacterial cells, yeast cells, fungal cells, insect cells, nematode cells, plant or animal cells. Suitable animal cells include, but are not limited to HEK cells, HeLa cells, COS cells, and various primary mammalian cells. Cells contained in intact animals, including but not limited to nematodes, zebrafish (and other transparent or semi-transparent animals) and fruitflies, may also be used in the methods of the present invention. An animal model expressing a βarrestin-detectable molecule fusion protein throughout its tissues, or within a particular organ or tissue type, will be useful in studying cellular targets of known or unknown ligands in the Hh pathway. Where diagnosis is being assayed cell refers to an appropriate cell of the subject being test. So for example, the cell can be any cell of the body or can be a cell taken from a suspected cancerous tissue or the like.

The phrase “kinase capable of phosphorylating the Smoothened receptor” refers to any kinase which when in the presence of the Smoothened receptor and appropriate ion source can phosphorylate the intracellular portion of the Smo receptor. It has been recently discovered the GRK2 is capable of having that function however, in a test system any kinase which achieves that role would be acceptable and other kinases such as casein kinase are known to function as GRK type kinases. The phosphorylation readies the receptor for internalization by arrestin.

As used herein, “exposing” a cell to a test compound or solution means bringing the cell exterior in contact with the test compound or solution. Where the test compound or solution is being screened for Smoothened ligand activity or Hh pathway activity, exposure is carried out under conditions that would permit binding of a ligand to a receptor expressed in that cell. As used herein, “translocation” of arrestin refers to movement of the arrestin molecule from one area of the cell to another.

When a test compound is “active on the Smoothened receptor” it is meant that it either has a measurable agonistic activity or an antagonistic activity. It also refers to measurable activity that would qualify it as a partial agonist or partial antagonist. Determination as an agonist or antagonist can be accomplished by previously known methods for determining active compounds on GPCRs. Accordingly; the methods for determining activity of a test compound are disclosed in the “Duke patents” with the substitution of an Smo in place of a GPCR. Since Ptc inhibits Smo and it is known that Hh such as Shh removes that inhibition, measuring Smoothened activity is a measure of Hh, Ptc or directly the Smo. This would include the part of the pathway that activates the Smo translocation cascade of events including arrestin and Smo phosphorylating kinases. In other words a positive agonistic or antagonistic effect on the Smo could result from a test compounds effect anywhere from direct action on Smo and the Smo affector compounds itself upstream through the Hh protein ligand. Accordingly, active test compounds discovered with the assay of the invention would wind up being the same type of compounds discovered with the reporter gene assays previously known with less error and much faster.

An aspect of the present invention is a method of assessing Hh pathway activity under test conditions, by providing a test cell that expresses a Smoothened receptor and that contains a conjugate of an arrestin protein and a reporter molecule, for example, a visually detectable molecule; exposing the test cell to a known agonist under test conditions; and then detecting translocation of the detectable molecule from the cytosol of the test cell to the membrane edge of the test cell. Translocation of the detectable molecule in the test cell indicates activation of the Hh pathway. Exemplary test conditions include the presence in the test cell of a test kinase and/or a test Smo, or exposure of the test cell to a test ligand.

A further aspect of the present invention is a method of screening a test compound for Smo or Hh pathway antagonist activity. A cell is provided that expresses a Smo and contains a conjugate of an arrestin protein and a reporter molecule. The cell is exposed to a test compound and to a Smo agonist, and translocation of the reporter molecule from the cell cytosol to the membrane edge is detected. When exposure to the agonist occurs at the same time as or subsequent to exposure to the test compound, movement of the detectable molecule from the cytosol to the appropriate membrane edge after exposure to the test compound indicates that the test compound is not an antagonist.

A further aspect of the present invention is a method of screening a test compound for Smo or Hh pathway antagonist activity. A test cell is provided that expresses a Smo and contains a conjugate of an arrestin protein and a reporter molecule. The cell is exposed to an agonist (but this is not necessary if there is constitutive activity or an endogenous agonist available) so that translocation of the detectable molecule from the cytosol of the cell to the membrane edge of the cell occurs, and the cell is then exposed to a test compound. Where exposure to the agonist occurs prior to exposure to the test compound, movement of the detectable molecule from the membrane edge of the cell to the cytosol after exposure of the cell to the test compound indicates that the test compound has Smo or Hh pathway antagonist activity.

A further aspect of the present invention is a method to screen a test compound for Smo or Hh pathway agonist activity. A test cell is provided that expresses a Smo and contains a conjugate of an arrestin protein and a reporter molecule. The cell is exposed to a test compound, and translocation of the detectable molecule from the cell cytosol to the membrane edge is detected. Movement of the detectable molecule to the membrane edge after exposure of the cell to the test compound indicates Smo agonist activity of the test compound. The test cell may express a known Smo.

A further aspect of the present invention is an apparatus for determining Smo activity in a test cell. The apparatus includes means for measuring indicia of the intracellular distribution of a detectable molecule, and a computer program product that includes a computer readable storage medium having computer-readable program code means embodied in the medium. The computer-readable program code means includes computer-readable program code means for determining whether the indicia of the distribution of the detectable molecule in a test cell indicates concentration of the detectable molecule at the cell membrane, based on comparison to the measured indicia of the intracellular distribution of a detectable molecule in a control cell. The indicia of the intracellular distribution of the detectable molecule may be optical indicia, and the measuring means may be means for measuring fluorescent intensity. The molecule to be detected may be one that is fluorescently detectable, and the step of measuring the indicia of the intracellular distribution of the detectable molecule may include measurement of fluorescence signals from test and control cells.

A further aspect of the present invention is an apparatus for determining Smo activity in a test cell. The apparatus includes means for measuring indicia of the intracellular distribution of a detectable molecule in at least one test cell at multiple time points, and a computer program product. The computer program product includes a computer readable storage medium having computer-readable program code means embodied in said medium. The computer-readable program code means includes computer-readable program code means for determining whether the indicia of the distribution of the detectable molecule in the test cell at multiple time points indicates translocation of the detectable molecule to the cell membrane.

A further aspect of the present invention is an apparatus for determining Smo activity in a test cell, which includes means for measuring indicia of the intracellular distribution of a detectable molecule in at least one test cell, and a computer program product. The computer program product includes a computer readable storage medium having computer-readable program code means embodied therein and including computer-readable program code means for determining whether the indicia of the distribution of the detectable molecule in the test cell indicates concentration of the detectable molecule at the cell membrane, based on comparison to pre-established criteria.

An automated high throughput system for detecting compounds using the novel information and assay of the invention can be done by intercellular measurement of the translocation of the receptor molecule arrestin conjugate. Such a method is disclosed in the “Duke patents” and by substitution of the Smo for any GPCR a high throughput screening assay as claimed is provided. Other reporter molecule methods are well known in the art including the BRET and FRET assays. Phosphorylation of Smo is a mechanism leading to internalization of the receptors; receptors that have been stimulated redistribute, whereas the responses of other receptors remaining on the cell membrane remain intact. Arrestin dependent binding is induced only when the Smo is activated. Various isoforms of arrestin are known in addition to those described above; arrestin further refers to all such isoforms of arrestin, proteins having substantial sequence similarity thereto which are functional arresting, and functional fragments thereof. Functional fragments of arrestin, its isoforms and analogs, may be determined using techniques as known in the art.

The present inventors have determined that arrestin redistribution (translocation) to the cytosol, to the cell outer plasma membrane, to vesicles and to other locations in the cell, occurs in response to activation of the Smo, including ligand and non-ligand activation. The present inventors demonstrated a common role for arrestin in mediated signal transduction following activation of receptors. The present inventors have devised convenient methods of assaying activation of Smo in vivo and in vitro in real time. The methods of the present invention utilize arrestin translocation to provide a single-step, real-time assessment of Smo function. The present methods may additionally be surprisingly utilized in studying and understanding the mechanisms of actions of various therapeutic agents i.e. molecules which act as agonists or antagonists in the Hh pathway (including partial agonists and antagonists). The present inventors have determined that a protein conjugate or chimera comprising an arrestin molecule and a reporter molecule (such as Green Fluorescent Protein) is useful in such methods of assaying in vivo and in vitro Smo activity especially as an assay for screening test compounds either individually or in high throughput screens.

Cells used in the testing as described above can either have the normal, natural amounts of kinase and Smo or can be manipulated to include an increased amount of these as well as the arrestin-reporter conjugate. These determinations as well as the methods for making and using them are well known in the art of GPCR assay and can be utilized herein substituting a Smo where appropriate.

Since it has been determined that an increase or decrease in Smo signaling from a normal state as a pre-established criteria, (i.e. some interruption in the Hh pathway in that individual) creates a medical condition such as cancer, a screen of an individual would be useful in detecting or predicting the onset of disease. Accordingly, healthy subjects could be tested for a predisposition toward disease or a particular tissue cell tested to see if it is exhibiting an indicator of disease. So, for example, a cell from a test subject could be collected and transformed with a reporter arrestin conjugate. In addition, to make sure any defect is not in the production or availability of kinase, the cell could be transformed with a kinase that is capable of phosphorylating a Smo, such GRK2.

Once the appropriate transformed cell is collected the cell can be contacted with a known ligand of the Smo. These ligands are well known such as cyclopamine, but the background art lists hundreds more. Once the ligand of the Smo is contacted with the Smo, the assay of the invention can be used to measure in a quantitative way the activity of the Smo. Once that measurement is taken the result can be compared to a pre-established activity criteria with that ligand, for example doing the same assay with cells known to be free from defect or medical condition. By observing if the test results are higher or lower than the pre-established activity criteria one can determine if the individual is or will be susceptible to a disease or have a disease that result from over or under signaling by the Smo.

It has been also found that in a cell that cancerous or over proliferating, there will be an increased number of Smos present on the surface. By determining if any tissue, cell or location that has an increased number of Smo compared to a normal value as a pre-established criteria, not only can the presence and location of a cancerous tumor be located, so can its size and shape. Accordingly, by comparing the amount of active Smoothened receptors in the test subject a cell or cells with an elevated number of receptors compared to a pre-established criteria for those types of cells would indicate either a predisposition to the development of cancer or actual cancerous state. By determining the size, the location or the shape of a tumor, those skilled in the art can determine the effectiveness of cancer treatments by monitoring the modulation in size and shape of a known tumor

Using the methods of this present invention then, a conjugate of a Smo ligand and a reporter molecule are administered (for example orally or injectably) to a test subject or a test tissue from the subject. The ligand is allowed time to distribute through out the body or tissue. The reporter molecule is then assayed for location and quantity for example, by radioactive reporter assay. By observing any area where the conjugate accumulates and then noting which if any, areas have an over accumulation of conjugate compared to a pre-determined criteria, i.e. normal cell distribution, on can determine that the area with an over abundance of Smos are cancerous tissue.

The present invention is explained in further detail in the following examples which are intended to be non-limiting in scope. One skilled in the art, in view of the disclosure herein could readily substitute in these examples and provide alternative methods.

### EXAMPLES

### Materials and Methods

Materials were obtained from commercially available sources or made using known techniques from known starting materials, including the Smo, the Hh plasmid and the Ptc plasmid.

### Example 1

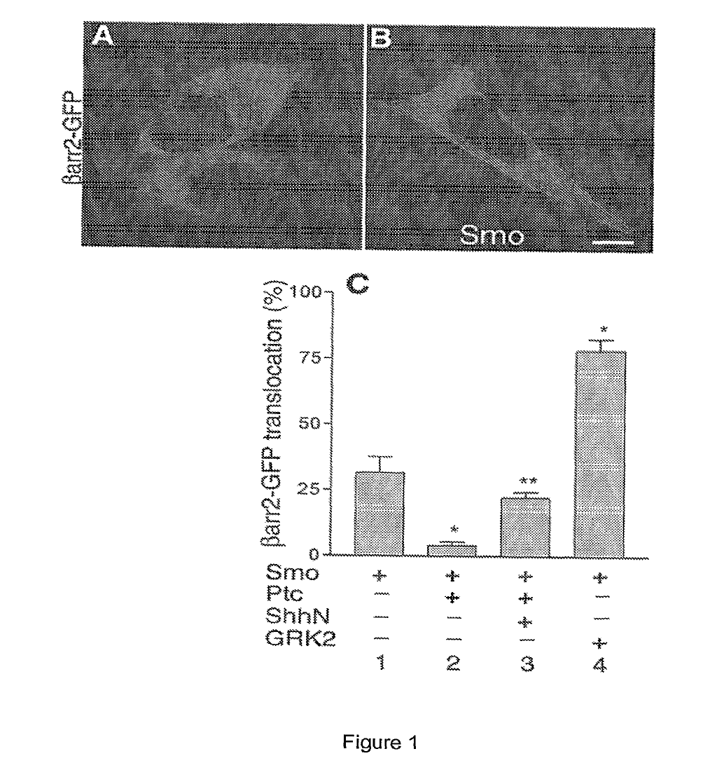

Localization of beta-arrestin2 green fluorescent protein (βarr2-GFP) to the plasma membrane in cells overexpressing Smo seen in FIG. 1: Confocal images of βarr2-GFP expressed alone (A) or with Myc-Smo (B) in Human embryonic kidney (HEK)293 cells. (C) Effects of Ptc, ShhN, and G protein coupled receptor kinase (GRK2) on recruitment of βarr2-GFP to the plasma membrane. βarr2-GFP was expressed with Myc-Smo (bar 1), Myc-Smo and FLAG-Ptc (bar 2), Myc-Smo and FLAG-Ptc (co-cultured with HEK293 expressing Sonic hedgehog (Shh)N-terminus, an active form of Shh (bar 3), or Myc-Smo and GRK2 (bar 4) in HEK293 cells. Data are presented as the percentage of βarr2-GFP-13 expressing cells with recruitment of βarr2-GFP and are the means±SEM of three independent experiments. *P<0.05 (compared with bar 1) and **P<0.005 (compared with bar 2) (unpaired t test). Scale bar, 10 μm.

### Example 2

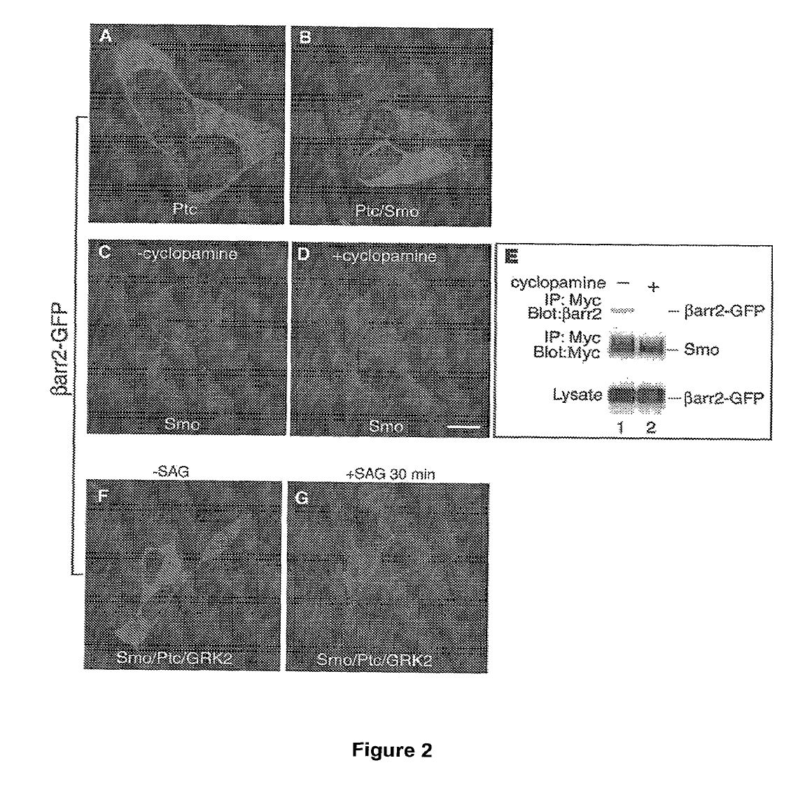

Ptc and cyclopamine inhibit membrane recruitment of βarr2-GFP, and SAG relieves Ptc inhibition of recruitment of βarr2-GFP to the plasma membrane as shown in FIG. 2. Confocal images of βarr2-GFP expressed with FLAG-Ptc (A), FLAG-Ptc and Myc-Smo (B), and Myc-Smo (C and D) in HEK293 cells. Cells were left untreated (A to C) or treated with 8 μM cyclopamine (D) at 37° C. for 5 min. Recruitment of βarr2-GFP to Smo was ablated by treatment with cyclopamine (C versus D), but not with dimethylsulfoxide (DMSO), a vehicle for cyclopamine. (E) Effect of cyclopamine on interaction of βarr2-GFP with Smo. HEK293 cells stably expressing Myc-Smo and βarr2-GFP were left untreated (lane 1) or treated with cyclopamine (6 μM, lane 2) at 37° C. for 1 hour. Cell extracts were immunoprecipitated with anti-Myc affinity gel. Immunoprecipitates were immunoblotted with antibodies against βarr2 (A2CT) (top) or antibodies against Myc (middle). Whole-cell lysates were immunoblotted with A2CT antibodies (bottom). (F and G) Confocal images of cells stimulated with 0.3 μM SAG at 37° C. for 0 min (F) and 30 min (G). HEK293 cells were transfected with βarr2-GFP, FLAG-Ptc, Myc-Smo, and GRK2. Scale bar, 10 μm. Representative images or a blot of three independent experiments are shown.

### Example 3

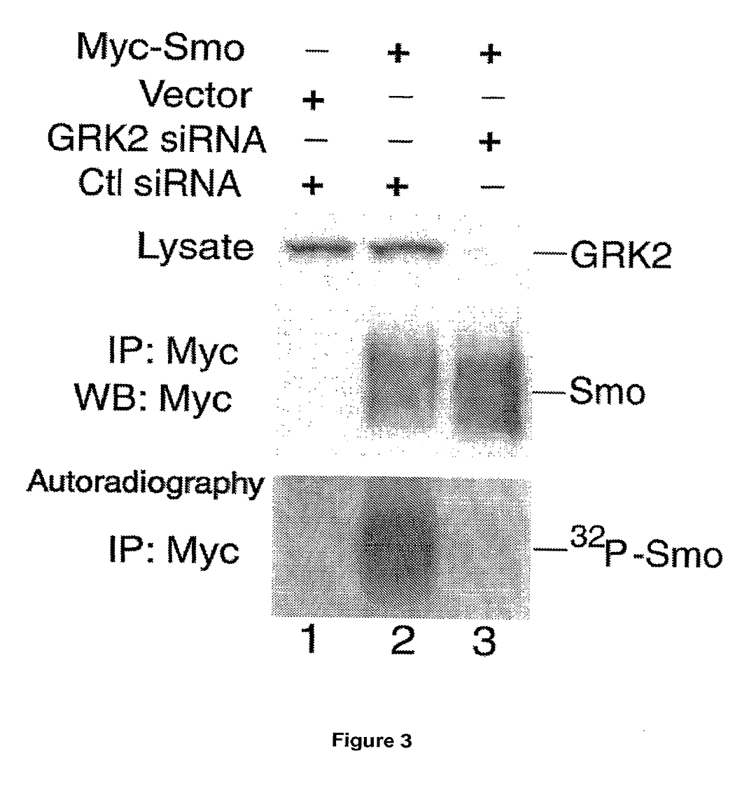

Phosphorylation of Smo mediated by GRK2 shown in FIG. 3. Decreased phosphorylation of Smo in cells lacking GRK2. HEK293 cells were transfected with control siRNA and DNA empty vector (lane 1), or Myc-Smo (lanes 2 and 3) along with control siRNA (lane 2) or siRNA directed against GRK2 (lane 3). Cells were incubated with [³²P]orthophosphate at 37° C. for 1 hour. Proteins from cell extracts were either immunoblotted with antibodies against GRK2 (top) or immunoprecipitated with anti-Myc affinity gel. Immunoprecipitates were either immunoblotted with antibodies to Myc (middle) or processed for autoradiography (bottom). A representative blot of three independent experiments is shown.

### Example 4

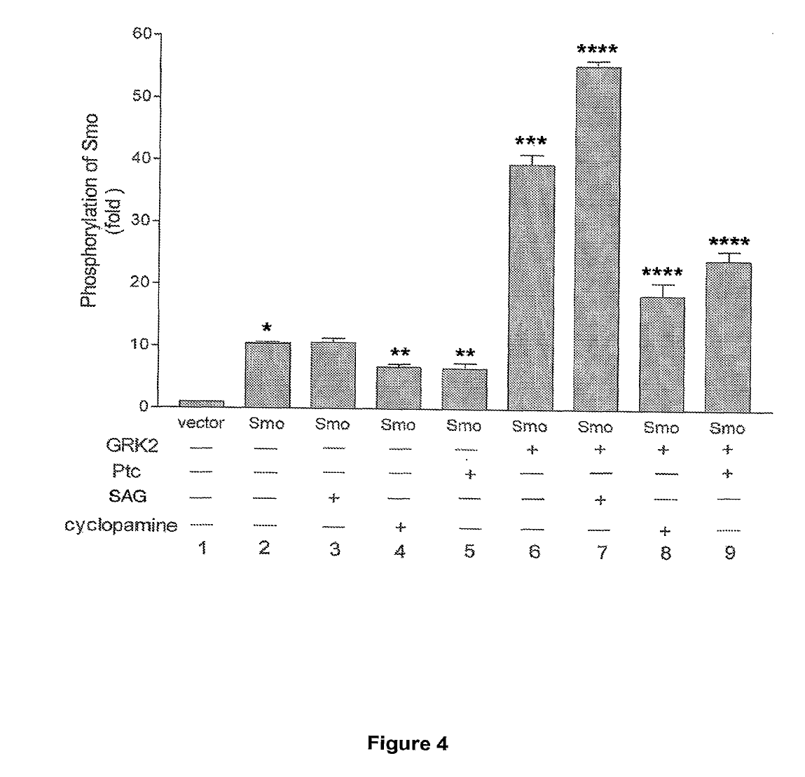

Effects of GRK2, Ptc, SAG, and cyclopamine on Smo phosphorylation shown in FIG. 4. HEK293 cells were transfected with vector (bar 1), or Myc-Smo (bars 2 to 9), and FLAG-Ptc or GRK2 (bars 5 to 9) as indicated. Cells were labeled with [³²P]orthophosphate at 37° C. for 1 hour and then left untreated or treated with 0.3 μM SAG (bars 3 and 7) or 2 μM cyclopamine as indicated at 37° C. for 15 min. Proteins from cell extracts were immunoprecipitated with anti-Myc affinity gel. Immunoprecipitated Smo was processed for autoradiography. Data are presented as fold changes of Smo phosphorylation over that in cells transfected with control vector. *P<0.005 (compared with bar 1); **P<0.02 and ***P<0.0001 (compared with bar 2); ****P<0.005 (compared with bar 6) (unpaired t test). The results shown are the means±SEM of three independent experiments.

### Example 5

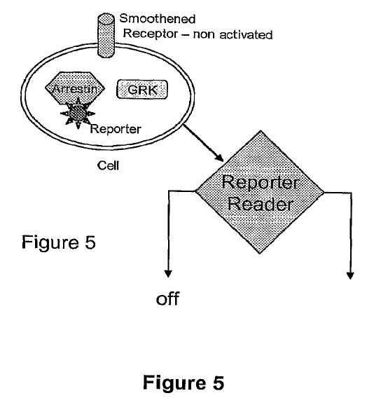

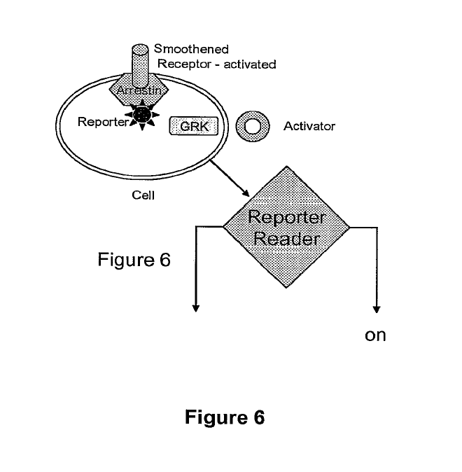

FIGS. 5 and 6 depict an active and an inactive Smo. The two graphic representations show the activity pattern involved in both an active an inactive Smo.

### Example 6

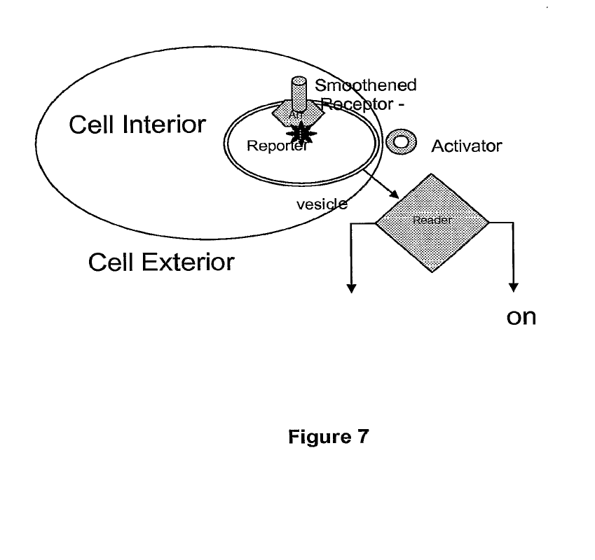

FIG. 7 shows the translocation of the Smo from the cell membrane to the membrane of a vesicle

### Example 7

### Testing Unknown as an Smo Ligand

Apply 10 microliters of a 250 micromolar concentration of unknown ligand to the well containing the cells expressing Smoothened receptor and arrestin-GFP in a volume of 100 microliters of media. An antagonist will change the distribution of beta-arrestin from membrane or aggregated to a uniform cytoplasmic distribution over a period of a few hours at room temperature to 37 degrees.

Examples and the explanations herein are intended to be illustrative of the present example on not to be construed as limiting in any manner. The invention is defined by the following claims with equivalents to each claim and part of the claim intended to be included therein.

What is claimed is:

1. A method for screening a test compound for Smoothened receptor activity comprising:
a) providing one or more cells that expresses a Smoothened receptor and contains a conjugate of arrestin and a reporter molecule, and also contains a kinase capable of phosphorylating the Smoothened receptor;
b) exposing the one or more cells to a test compound; and
c) determining if the test compound is active on the Smoothened receptor activity.

2. A method according to claim 1 wherein step c) is for determining if the test compound is an antagonist.

3. A method according to claim 1 wherein step c) is for determining if the test compound is an agonist.

4. A method according to claim 1 wherein the arrestin is β-arrestin.

5. A method according to claim 4 wherein the β-arrestin is β-arrestin2.

6. A method according to claim 1 wherein the reporter molecule is a visually detectable molecule.

7. A method according to claim 6 wherein the visually detectable molecule is a fluorescent protein.

8. A method according to claim 1 wherein the kinase is GRK2.

9. A method according to claim 1 wherein the Smoothened receptor is over expressed.

10. A method according to claim 1 where in the method is used to screen for compounds having an activity selected from the group comprising proliferative, antiproliferative and anticancer activity.

11. A method according to claim 1 which further comprises an automated apparatus and computer program means for measuring the intracellular location of the reporter molecule.

12. A method according to claim 1 which further comprises an automated apparatus and computer program means for measuring the intracellular intensity of the reporter molecule.

13. A method for diagnosing the cause of a medical condition mediated by the Smoothened receptor wherein the medical condition is mediated by a deviation in the Smoothened receptor activity from a pre-established activity criteria comprising:
a) providing a cell from a test subject or tissue which has been transformed to contain a conjugate of arrestin and a reporter molecule and which cell contains a kinase capable of phosphorylating the Smoothened receptor;
b) exposing the cell to a known ligand of the Smoothened receptor;
c) determining the activity of the Smoothened receptor in the cell; and
d) comparing the activity in the cell to the pre-established activity criteria.

14. A method according to claim 13 wherein the medical condition is cancer.

15. A method according to claim 13 wherein the kinase is GRK2.

16. A method for the diagnosis of cancer in a subject comprising:
a) administering to a subject or one or more cells from a subject, a conjugate of a ligand of the Smoothened receptor and a reporter molecule;
b) quantitatively determining the location or intensity of the reporter molecule in the subject or cell;
c) determining the presence of cancer where ever there is an accumulation of reporter molecule that is above a pre-established criteria.

17. A method of diagnosis according to claim 16 wherein the accumulation of the reporter molecule that is above a pre-established criteria is used to determine the size of a cancerous growth or tumor.

18. A method according to claim 16 wherein the reporter molecule is a radioactive reporter molecule.

19. A method according to claim 16 wherein the Smoothened ligand is a high affinity antagonist of the Smoothened receptor.

20. A method according to claim 16 wherein the Smoothened ligand is a high affinity agonist of the Smoothened receptor.

21. A method according to claim 16 wherein the presence of pancreatic or ovarian cancer is being detected.
